(19) United States
(12) Patent Application Publication
(10) Pub. No.: US 2007/0282629 A1
(43) Pub. Date: Dec. 6, 2007

(54) Patient information and communication system

(76) Inventor: Norbert Plambeck, Cuxhaven (DE)

Correspondence Address: HARNESS, DICKEY & PIERCE, P.L.C., P.O. BOX 828, BLOOMFIELD HILLS, MI, 48303, US

(21) Appl. No.: 11/445,501

(22) Filed: Jun. 1, 2006

(51) Int. Cl. G06Q 10/00; A61B 5/00

(52) U.S. Cl. 705/2; 600/300

(57) ABSTRACT

A method of compiling and utilizing patient information includes receiving patient data related to a particular patient, storing the patient data and generating a patient medical history including the patient data. The method further includes providing a search filter having a plurality of search fields, communicating between the patient medical history and the search filter, wherein patient data from the patient medical history is directly input into at least one of the search fields, and searching an information source using the search filter.

### FIELD

The present disclosure relates to information compilation and distribution, and more particularly to a system for compiling, summarizing, distributing and searching patient information.

### BACKGROUND

The statements in this section merely provide background information related to the present disclosure and may not constitute prior art.

A patient's medical history (PMH) is one of the most important records that is collected and maintained over a patient's lifetime. The PMH provides information that can be used for current patient treatment, to monitor the progression of health indicators during the patient's lifetime, as well as to diagnose and/or inhibit illnesses. A patient may visit his/her doctor regularly (e.g., family doctor), as well as visiting other doctors or health care specialists for specialized treatment. For example, although a patient may visit a family doctor for annual check-ups (e.g., annual physical) and to treat short-term illnesses (e.g., a cold), the patient may also periodically visit and be treated by, for example, a dermatologist, an ear, nose and throat specialist, a cancer specialist, a physical therapist, or other specialist.

Each of the medical professionals that treat a patient maintains his/her own records, which may not be readily accessible to the patient. Further, because such records are designed for use and interpretation by trained medical professionals, even if a patient is provided a copy of his/her medical record, it is often impossible for the patient to fully understand the information, and the significance of the information to the patient's well-being is often lost.

In many cases, the medical records of one medical professional may not be accessible to another medical professional that is concurrently treating the patient. In such cases, the onus is on the patient to accurately convey the PMH. This often occurs during an initial visit to a new doctor, whereby the patient fills out a medical history form. The patient may inaccurately and/or incompletely provide information that is significant to the diagnosis and treatment of the patient, which could lead to misdiagnosis and/or mistreatment of the patient.

In some cases, a plurality of patient's may be afflicted with a common ailment. One patient may be undergoing a specific treatment or therapy, while another patient may be undergoing an alternative treatment or therapy, in particular, if the second patient is treated by a different doctor or health care professional. Currently, patients are left to using general search tools, such as the Internet, to research alternative treatments that may be better for their particular situation. Although the patient's doctor is also a source for information regarding treatment options, the doctor's suggestions may not be as objective as desired, and may be biased toward a particular treatment that the patient's doctor personally prefers or specializes in.

### SUMMARY

Accordingly, the present invention provides a method of compiling and utilizing patient information. The method includes receiving patient data related to a particular patient, storing the patient data and generating a patient medical history including the patient data. The method further includes providing a search filter having a plurality of search fields, communicating between the patient medical history and the search filter, wherein patient data from the patient medical history is directly input into at least one of the search fields, and searching an information source using the search filter.

In another feature, the information source includes a medical database that contains information related to known illnesses, symptoms, treatment options, specialists, doctors, treatment costs, and the like.

In another feature, the method further includes automatically executing the step of searching upon completion of the step of storing the patient data.

In other features, the method further includes receiving additional patient data related to a plurality of patients, storing the patient data and the additional patient data in a database, and providing information to enable a comparison between a treatment program of the particular patient to that of at least one anonymously identified patient based on the patient data and the additional patient data. The method also includes providing information associated with at least one health care professional that is treating the at least one anonymously identified patient.

In other features, the method further includes generating summaries of the patient data for the particular patient. The summaries include at least one of a graph and a table.

In another feature, the method further includes generating an alert based on the patient data, the alert indicating an undesired health condition.

In another feature, the method further includes generating health indicator statistics based on patient data that is associated with a plurality of patients and that is stored within the database.

In still another feature, the method further includes providing a plurality of access levels, one of which is assignable to each of a plurality of authorized users. The access levels define which patient data is accessible to a particular authorized user.

In yet another feature, the method further includes communicating the patient data to a health care provider.

In general, a patient information and communication system (PICS) is provided that enables a user to gather, archive and process patient related data to create an electronic file or patient medical history (PMH) for a particular patient. The PICS simplifies communication between a patient, a treatment specialist (e.g., doctor or therapist) and other parties including, but not limited to, a health insurance provider. The PICS further enables a user to acquire relevant health related information and provides a personal search filter (PSF) that enables a user to more effectively search information sources. The PICS of the present invention includes, but is not limited to, the following components: the PMF, a communications function, at least one database and the PSF. The PMH is also provided to the patient in an easy to understand format including tables, charts and graphical summaries that are provided in clear terms, understandable by the layman.

Advantageously, the PMH is a patient-specific personal file for the collection and documentation of all health related data, and is the basis of the PICS. The PMH provides a concise description and evaluation of the health related values contained therein. The PMH is accessible by a user (e.g., the specific patient) via the PICS program running on any one of the user interface devices, whereby the program's user interface enables user access to a plurality of system tools.

A medical database is advantageously provided and includes information relating to all known illnesses, symptoms, treatment options, specialists, doctors, treatment costs, and the like. The medical database is searchable using the PSF and provides explanations in terms that the laymen would understand, as well as providing more detailed medical explanations in any one of a number of languages. The medical database further provides objective descriptions of current medical opinions related to a specific topics, as well as objective information about the treatment options, doctors, specialists and/or alternative treatments contained therein.

The PSF is used to search an information source or sources for information relevant to a particular patient and enables non-relevant data to be filtered out, such that only information that is relevant to a particular patient is provided. As a result, the PSF provides patient-specific search results that are focused, thereby reducing the amount of post-search reading and filtering that a searching user would otherwise be required to perform his/her self to identify information that is especially relevant and useful.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 illustrates an exemplary communication network that is implemented by the patient information and communication system (PICS) of the present invention;

FIG. 2 illustrates an exemplary search network that is accessible using the PICS of the present invention; and

FIG. 3 illustrates a schematic, exemplary patient information summary that is generated by the PICS of the present invention.

### DETAILED DESCRIPTION

The following description is merely exemplary in nature and is not intended to limit the present disclosure, application, or uses.

As explained in detail herein, the present invention provides a patient information and communication system (PICS) that enables a user to gather, archive and process patient related data to create an electronic file or patient medical history (PMH) for a particular patient. The PICS simplifies communication between a patient, a treatment specialist (e.g., doctor or therapist) and other parties including, but not limited to, a health insurance provider. The PICS further enables a user to acquire relevant health related information and provides a personal search filter (PSF) that enables a user to more effectively search information sources. The PICS of the present invention generally includes the following components: the PMF, a communications function, at least one database and the PSF. Each of these components is described in further detail below.

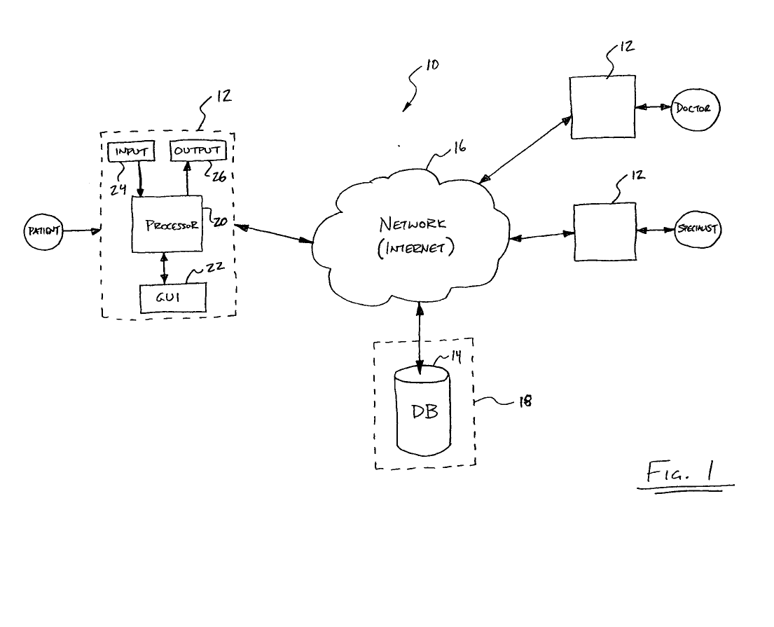

Referring now to FIG. 1, an exemplary communication network 10 is illustrated. The communication network 10 includes a plurality of user interface devices 12 that communicate with a database 14 over a network 16, such as the Internet. The database 14 can be resident in the memory of one of the user interface devices 12 or can be resident in the memory of a third-party server 18. Each user interface device 12 includes a device or processor 20 that enables communication with and data transfer over the network 16. Exemplary user interface devices 12 include, but are not limited to, a computer, a personal digital assistant (PDA), a mobile phone and any other device that connects with and enables communication over the network 16. The user interface device 12 preferably includes a graphical user interface (GUI) 22 that displays requested data. The user interface device 12 further includes an input device 24 including, but not limited to, a keyboard, a mouse, a touch-pad or a touch-screen (e.g., touch-screen GUI). The input device 24 is used to navigate the network 16, to access the database 14 and to input additional information. An output device 26, such as a printer, is also provided to produce hard copies of information that is accessed using the user interface device 12.

It is further anticipated that the output can be provided in electronic, paperless formats including, but not limited to, email and/or text messages, or any other form of electronic document that is communicated to a user. For example, electronic messages can be automatically generated and delivered in order to provide specific information to a specific user or users, upon the information being integrated into the PMH, as explained in further detail below. It is also anticipated that the electronic message can include hyper text mark-up language (HTML) links that enable an authorized user (e.g., a patient authorized doctor or health care provider) to access the patient-related information. For example, the authorized user is required to input his/her user information before the link accesses the patient-related information. In this manner, the patient-related information is not directly provided in the electronic message itself.

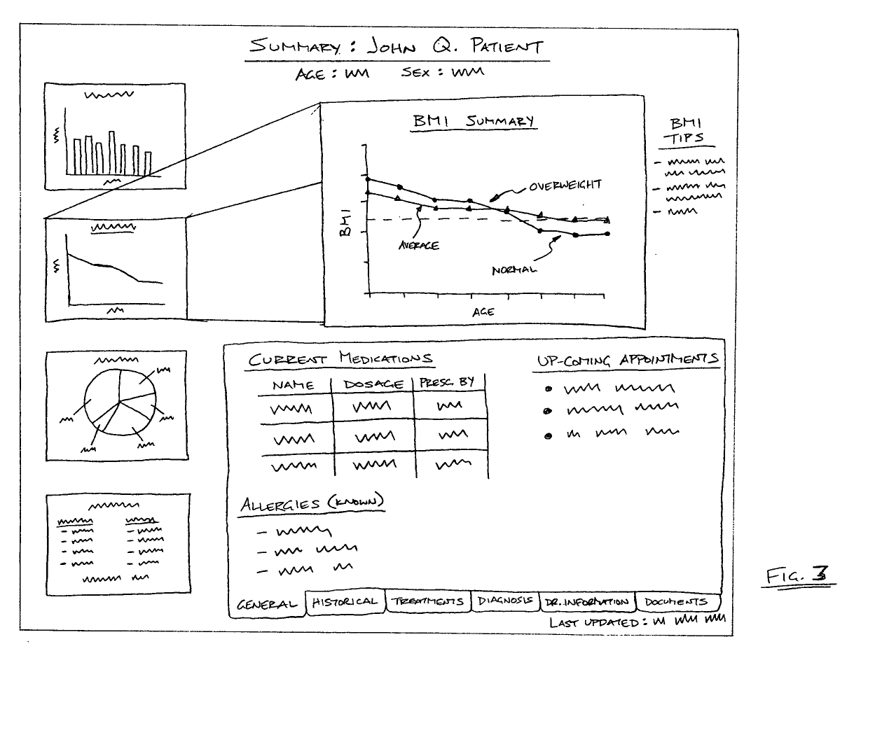

The PICS of the present invention is preferably implemented over the communications network 16 and is executed using software that is resident on one or more of the user interface devices 12. More specifically, the PICS provides a portal for a user to access patient-specific information that is stored in the database 14, to input new information into the database 14 and/or to update or edit information that is stored in the database 14. The information that is stored in the database 14 is compiled and summaries or reports are generated, which are readily understandable by the layman (e.g., a person that is not a medical professional). The PICS also generates reports and/or summaries that are more technically oriented for use by medical professionals in patient treatment. As schematically illustrated in FIG. 3, the PICS can present patient information in tabular and graphical form, to enable easy recognition of trends. For example, trends in health indicators including, but not limited to, blood pressure, blood sugar, insulin, cholesterol (i.e., total cholesterol including the high-density lipoproteins (HDL) and the low-density lipoproteins (LDL)), weight, height and body mass index (BMI) are readily recognizable in graphical form.

It is also anticipated that the PICS provides an alert for an unacceptably high or low health indicator value, as well as providing recommendations to increase/reduce the health indicator to an acceptable range. For example, the PICS issues an alert if the patient's BMI is within an overweight or obese range, whereby the alert for the obese range is more dramatic than that for the overweight range. Concurrently, the PICS provides approved tips or advice (i.e., approved by a recognized medical body) for safely reducing/increasing the value of the health indicator. In the case of high cholesterol, for example, the PICS may make suggestions on particular foods that may help in lowering the cholesterol level, or may suggest that the patient speak to his/her doctor about cholesterol reducing medications. The PICS also provides average patient values for comparison purposes. In the case of weight or BMI, for example, the PICS provides the average weight and/or BMI for people of the same age and height as the patient.

The PICS further compiles and provides a complete electronic file or patient medical history (PMH). The PMH includes complete health information from birth up through death and even includes digital copies of important documents including, but not limited to, a birth certificate, x-ray images, a death certificate and an autopsy report. The PMH further includes information such as current and past medications, family medical history, whether the patient is post-menopausal, whether or not the patient is a smoker or drinker, and if so, from what age and at what rate (e.g., cigarettes and/or alcoholic drinks per day), as well as diagnosis and treatment summaries for past or current illnesses experienced by the patient. The PMH is periodically updated throughout the patient's lifetime, as discussed in further detail below. In this manner, the PICS provides up to date patient information and a complete PMH.

In general, the PMH is a patient-specific personal file for the collection and documentation of all health related data, and is the basis of the PICS. The PMH provides a concise description and evaluation of the health related values contained therein. The PMH is accessible by a user (e.g., the specific patient) via the PICS program running on any one of the user interface devices, whereby the program's user interface enables user access to a plurality of system tools.

Each of the plurality of user interface devices is accessible by respective authorized users. At least one of the authorized users includes a patient and/or a patient guardian that is legally responsible for the well-being of the patient in the event that the patient is incapacitated (e.g., mentally or physically) or is otherwise unable to use the user interface device. Other users can include the patient's family doctor, dentist or a specialist doctor, as well as an authorized person employed at a clinic or institute, at which the patient is being treated.

Each user is assigned a user-specific identification and password combination, which is associated with an access level. For example, the patient's and/or family doctor's identification and password combination provides full access to all information accessible using the PICS. The patient's identification and password combination, however, can restrict which information is editable or updatable by the patient. For example, only the family doctor or specialist doctor is able to edit or update information including, but not limited to, medication, treatment history and/or health indicator values. In this manner, the PICS inhibits the patient, who may not be a medical professional, from inadvertently altering critically important health information. Other users may only have access to very specific information. For example, if the patient is being treated at a physical therapy clinic, the patient's physical therapist may have access to information pertaining only to the physical therapy, and only for the duration of the physical therapy. The access level for the particular users can be set by the patient and preferably by the family doctor, who is more aware of which users require what information.

The PICS enables automatic updating of the patient's medical history. More specifically, the PICS provides visit-specific forms that include data input fields. For example, a form that is specific to a routine check-up or a visit to a dentist will have different data input fields than a form that is specific to a pre-surgery consultation. During the visit/consultation to the non-patient user (e.g., family doctor, specialist doctor, doctor's aid, nurse or nursing assistant, dentist) inputs the observed health indicators, diagnosis or any other relevant information directly into the user interface device. This information can be directly input into the form data input fields by the non-patient user as the information is collected or observed, or can be subsequently input based on notes taken during the visit/consultation. The information is subsequently uploaded to the database. The upload can occur upon command or can be scheduled to occur periodically (e.g., daily, weekly or monthly). It is preferred that the patient information be uploaded to the database at the earliest possible convenience so that it is readily accessible to other users.

It is further anticipated that the latest patient information be automatically delivered to the patient. For example, in the case where the doctor receives the results from tests that are carried out by an external laboratory (e.g., blood tests, DNA testing, etc.), the results of these tests are input into the PICS, which automatically generates a message that is delivered to the patient. This information is preferably delivered via an electronic message, and again is provided in clear terms that are readily understandable to the layman. In the case where the PMH is resident on the patient's user interface device, the PMH is automatically updated based on the information provided in the electronic message. In this manner, the information is provided paperless, and the PMH is updated without additional effort on the patient's part (i.e., without requiring the patient to manually update the PMH). This avoids the situation where the results may be lost and/or unused.

In the event that the patient is visiting a new family doctor or a specialist that does not yet have the patient's PMH, the PICS can be accessed prior to the patient's arrival and/or during the visit/consultation. In this manner, the onus is not on the patient to accurately and thoroughly fill in forms while sitting in the waiting room. As a result, the new family doctor or specialist has ready access to relevant and accurate patient information. In this manner, misdiagnosis and/or mistreatment are avoided because inaccuracies and incomplete information provided directly by the patient are avoided. Further, the PICS can be used to selectively filter the patient information so that the doctor can focus on the patient information that is particularly relevant to a specific medical situation. For example, if the patient is visiting a specialist to diagnose and treat a torn ligament, any patient information that is not relevant to such diagnosis and treatment can be filtered out to provide a concise summary of the relevant information.

As discussed above, it is anticipated that the database can be resident in memory of one of the user interface devices. For example, the PICS can be executed using a computer program that is resident on the patient's user interface device and that accesses the database, which is resident in memory of the patient's interface device. In such a situation, the patient can update the database information by uploading information from his/her doctor after a visit/consultation. Alternatively, the patient's doctor can electronically convey or otherwise provide the patient with an update file, which can be uploaded to the database. It is also anticipated that the patient can forward the patient information to a new doctor or new specialist prior to the patient's visit, so that the new doctor or specialist has the complete PMH before consulting with the patient. Alternatively, the patient can bring an electronic file containing the PMH to the doctor's office. In the event that the database is resident on the doctor's user interface device, the database is accessible by the patient so that the patient can obtain a complete summary of his/her medical history.

It is principally possible that the PICS can be maintained and managed by a third-party. That is to say, the third-party maintains the database, server and network access. Access and use of the PICS can be provided by the third-party gratis or for a fee. For example, the third-party could charge a yearly fee or a monthly fee to the patient, doctor and/or health care provider (i.e., insurance).

In the event that the PICS is maintained by a third-party or a doctor, it is anticipated that the database will include information for a plurality of patients (e.g., 10's, 100's or 1000's of patients). In such a scenario, the PICS enables calculation of averages and trends of health indicators or diagnosis across patients, while maintaining the confidentiality of the individual patients. More specifically, the PICS can extract data from the database to provide non-patient specific statistical summaries. For example, the average BMI, blood pressure, cholesterol level, etc. can be determined for patients of a particular age. In this manner, the doctor or third-party can observe overall trends for a particular group of patients. Further, the averaged values can be provided to the patient in his/her summary to compare their specific health indicators to the average.

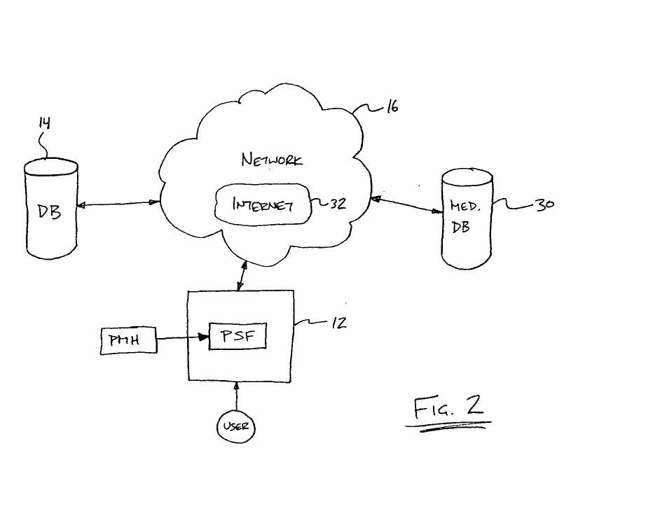

The PICS of the present invention also incorporates a medical database 30 (see FIG. 2) that is structured like a medical encyclopedia. More specifically, the medical database 30 includes information relating to all known illnesses, symptoms, treatment options, specialists, doctors, treatment costs, and the like. The medical database 30 is searchable using the PSF, described in further detail below, and provides clear and concise results. More specifically, the medical database 30 provides explanations in terms that the laymen would understand, as well as providing more detailed medical explanations in Latin, for example, and/or a patient-specific language (e.g., English, German, French, Japanese, etc.). The medical database 30 further includes an objective description of the current medical opinions related to a specific topic, as well as objective information about the treatment options, doctors, specialists and/or alternative treatments contained therein. In the present invention, specifically in the medical database 30, it is preferred that the alternative treatments, like for example acupuncture are considered completely equally entitled in addition to the “classical” medical teaching. The alternative treatments can include those that are recognized and sanctioned by a licensed medical group. The medical database 30 is preferably updated on a regular basis and is specifically attended to by a third party. The third party can provide free access to the database 30 or charge a fee.

With particular reference to FIG. 2, the PSF is used to search an information source or sources for information relevant to a particular patient. More specifically, a searching user (i.e., patient user or non-patient user) can search the database 14, the medical database 30 or even a general information source 32, such as the Internet, using the PSF provided by the PICS. The PSF enables non-relevant data to be filtered out, such that only information that is relevant to a particular patient is provided. More specifically, the PSF accesses the PMH to filter or narrow the search results. For example, if PMH indicates that the patient is an adult male, any data that is particular to children or adolescents is filtered out, as well as any information that is particular to females. Other information that may be relevant but not part of the PMH (e.g., a field that requests that the search results not include information that is more than 2 years old), can be input into appropriate fields of the PSF. In general, the PSF provides patient-specific search results that are focused. As a result, the PSF reduces the amount of post-search reading and filtering that the searching user would otherwise be required to perform his/her self to identify information that is especially relevant and useful.

As further mentioned above, the information source can include the database 14, whereby the searching user can search the database 14 for information pertaining to other, anonymous patients that may have similar health related issues. For example, the PSF enables a searching user to input data into pre-determined fields to identify other patients within the database 14 that have the same or similar health issues, although the other patients' actual identities remain confidential. The results of such a search can include, but are not limited to, a diagnosis, a treatment schedule and a summary of the results of the treatment, as well as the name of a doctor, doctors or other treatment providers that practice such treatments. In this manner, the PICS provides a tool for patients to anonymously assist one another or to assist a non-patient user (e.g., doctor) in gathering objective information on treatments that may or may not be helpful in treating a particular ailment, as well as identifying other sources (e.g., other doctors or a clinic) of such treatments.

It is anticipated that the information provided by the PICS searching function is objective and describes the advantages and disadvantages of any particular treatment. For example, the information can identify a treatment that, although successfully treats the particular ailment, includes side-effects that are undesired or unacceptable to a particular patient. The information can also identify an alternative treatment to a particular ailment as well as the doctor or clinic that provides that treatment.

Although, when searching the database 14, the PICS maintains the anonymity of the patients in the database, it is anticipated that the search results can also include information as to whether a particular anonymous patient is a member of a support group, Internet-based (e.g., chat forum) or otherwise (e.g., periodic meetings) and the contact information for such a support group. In this manner, a patient that has searched the database for other patients suffering from similar ailments may be able to indirectly contact those patients via the support group, or to make contact with the support group itself for assistance through the their ailment.

It is further anticipated that the searches described immediately above can be executed manually by the searching user, or by the PFS automatically, based on the PMH of a particular patient. More specifically, a search can be manually initiated by the searching user or can be automatically initiated by the PICS. For example, upon the patient information being input into the database 14 or being updated, the PICS executes the search to identify particularly relevant information using any or all of the information sources, including anonymously identifying other patients suffering from similar illnesses from the database 14 or providing patient-specific information form the medical database 30.

Referring now to FIG. 3, an exemplary patient summary or PMH will be described. The exemplary patient summary of FIG. 3 is illustrative of that which is observable by the patient or other user using the GUI. A series of graphs (e.g., bar, line, pie, etc.) graphically illustrate trends in the patient's health indicators. Patient information is also provided in tabular form. Each graph and/or table is selectable and can be magnified to provide more specific detail. Depending on which graph or table is selected, and the value of the patient's corresponding health indicator, tips or advice are provided to identify ways that the patient can improve the value of the health indicator. Other information is also provided including, but not limited to, general information (e.g., current medications, allergies and up-coming appointments), historical information, treatments (e.g., current and past), diagnosis (e.g., current and past), doctor information (e.g., doctor and/or specialist contact information), as well as digital copies of documents (e.g., birth certificate). The desired information is viewed by selecting a desired tab. It is appreciated that the patient summary of FIG. 2 is merely exemplary in nature and that the patient summary can include various health-related information in various forms.

The information contained in the PMH that is provided by the PICS of the present invention can be used to interpret and to analyze a patient's health situation, as well as to provide recommendations on patient treatment. Such recommendations can also be automatically provided by or can be input into the PICS in an easy to understand format. It is further anticipated that a doctor can access and review the PMH using the PICS and provide a remote diagnosis and treatment recommendations if the patient is unable to be present at the doctor's location. Further, the patient can use the information provided by the PMH to perform his/her own research into alternative treatments, etc., which can be conveyed to the doctor to support the patient's treatment.

The PICS also enables health care provider (i.e., insurance) information to be input. This information can be input by the patient or can be input directly by the health care provider as an authorized user. In this manner, the information can be automatically updated to reflect changes in the patient's health care coverage. The PICS can use the health care provider information to alert the patient as to what treatments are covered under his/her health care program and any co-pays, deductibles or other costs that the patient may incur as a result of the treatment. Other information such as governmental assistance through Medicare, Medicaid or any other government sponsored health care program can be provided. The health care provider and governmental information can be provided for multiple countries in the case where the patient travels internationally, has health care providers or treatment specialists in different countries and/or is able to receive assistance from health care programs of different governments.

It is also anticipated that the PICS can be implemented as a general communications tool. More specifically, data stored in the database can be communicated to the health care provider to assist in determining the coverage for a particular treatment. For example, after a patient visits a doctor, the doctor's diagnosis and recommended course of treatment, in hand with a fee estimate, can be automatically conveyed to the health care provider. In this manner, the health care provider is immediately provided with complete and accurate information regarding the patient's ailment and treatment thereof, and can estimate and provide the necessary support (e.g., payment) for such treatment, as well as identify any co-pays or deductibles, for which the patient is responsible, prior to executing the treatment.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A method of compiling and utilizing patient information, comprising:
receiving patient data related to a particular patient;
storing said patient data;
generating a patient medical history including said patient data;
providing a search filter having a plurality of search fields;
communicating between said patient medical history and said search filter, wherein patient data from said patient medical history is directly input into at least one of said search fields; and
searching an information source using said search filter.

2. The method of claim 1 wherein said information source includes a medical database that contains information related to known illnesses, symptoms, treatment options, specialists, doctors, treatment costs, and the like.

3. The method of claim 1 further comprising automatically executing said steps of searching upon completion of said step of storing said patient data.

4. The method of claim 1 further comprising:
receiving additional patient data related to a plurality of patients;
storing said patient data and said additional patient data in a database; and
providing information to enable a comparison between a treatment program of said particular patient to that of at least one anonymously identified patient based on said patient data and said additional patient data.

5. The method of claim 4 further comprising providing information associated with at least one health care professional that is treating said at least one anonymously identified patient.

6. The method of claim 1 further comprising generating summaries of said patient data for said particular patient.

7. The method of claim 6 wherein said summaries include at least one of a graph and a table.

8. The method of claim 1 further comprising generating an alert based on said patient data, said alert indicating an undesired health condition.

9. The method of claim 1 further comprising generating health indicator statistics based on patient data that is associated with a plurality of patients and that is stored within said database.

10. The method of claim 1 further comprising providing a plurality of access levels, one of which is assignable to each of a plurality of authorized users, wherein said access levels define which patient data is accessible to a particular authorized user.

11. The method of claim 1 further comprising communicating said patient data to a health care provider.

12. A patient information and communication system for compiling, summarizing, distributing and searching patient information, comprising:
at least one user interface device that communicates with a network;
an information source; and
a software program that is operable on said at least one user interface device and that performs steps including:
receiving patient data related to a particular patient;
storing said patient data;
generating a patient medical history including said patient data;
providing a search filter having a plurality of search fields;
communicating between said patient medical history and said search filter, wherein patient data from said patient medical history is directly input into at least one of said search fields; and
searching said information source using said search filter.

13. The system of claim 12 wherein said information source includes a medical database that contains information related to known illnesses, symptoms, treatment options, specialists, doctors, treatment costs, and the like.

14. The system of claim 12 wherein said information is resident on one of a plurality of user interface devices.

15. The system of claim 14 wherein said one of said plurality of user interface devices is associated with a third party that is not one of a plurality of authorized users.

16. The system of claim 12 wherein said software program automatically executes said step of searching upon completion of said step of storing said patient data.

17. The system of claim 12 wherein said software program receives additional patient data related to a plurality of patients, stores said patient data and said additional patient data in a database, and provides information to enable a comparison between a treatment program of said particular patient to that of at least one anonymously identified patient based on said patient data and said additional patient data.

18. The system of claim 17 wherein said software program provides information associated with at least one health care professional that is treating said at least one anonymously identified patient.

19. The system of claim 12 wherein said software program generates summaries of said patient data for said particular patient.

20. The system of claim 19 wherein said summaries include at least one of a graph and a table.

21. The system of claim 12 wherein said software program generates an alert based on said patient data, said alert indicating an undesired health condition.

22. The system of claim 12 wherein said software program generates health indicator statistics based on patient data that is associated with a plurality of patients and that is stored within said database.

23. The system of claim 12 wherein said software program provides a plurality of access levels one of which is assignable to each authorized user, wherein said access levels define which patient data is accessible to a particular authorized user.

24. The system of claim 12 wherein said software program communicates said patient data to a health care provider.
